(19) United States
(12) Patent Application Publication
(10) Pub. No.: US 2020/0330040 A1
(43) Pub. Date: Oct. 22, 2020

(54) METHOD AND SYSTEM FOR DETECTING NOISE IN VITAL SIGN SIGNAL

(71) Applicant: VITA-COURSE TECHNOLOGIES (HAINAN) CO., LTD., Haikou, Hainan (CN)

(72) Inventors: Ruiqing MA, Haikou (CN); Jiwei ZHAO, Haikou (CN); Chuanmin WEI, Haikou (CN); Zhiyong WANG, Haikou (CN)

(73) Assignee: VITA-COURSE TECHNOLOGIES (HAINAN) CO., LTD., Haikou, Hainan (CN)

(21) Appl. No.: 16/760,402

(22) PCT Filed: Oct. 31, 2017
(86) PCT No.: PCT/CN2017/108681

§ 371 Date: Apr. 29, 2020

(51) Int. Cl. A61B 5/00; A61B 5/024; G16H 50/20; G16H 40/67

(52) U.S. Cl. CPC A61B 5/7203; A61B 5/02416; A61B 5/0402; G16H 40/67; G16H 50/20

(57) ABSTRACT

The present disclosure provides methods and systems for analysing a noise in a vital sign signal, including functions of acquisition, data storage, calculation and analysis, processing, result output etc. of a vital sign signal. The systems may calculate and analyse information obtained in the vital sign signal, especially noises, by using various algorithms, and analyse or process a calculation result and output an analysis result.

### TECHNICAL FIELD

The present disclosure relates to methods and systems for obtaining, processing, extracting, and analysing a vital sign signal, and in particular, to methods and systems for detecting and identifying a noise in a vital sign signal.

### BACKGROUND

Photoplethysmography (PPG) is a non-invasive detection method for detecting changes in blood volume in living tissues by means of photoelectricity. Basic physiological parameters of human bodies, such as a heart rate, a blood oxygen saturation, a respiratory rate, a blood pressure, etc. may be obtained by using the PPG. A PPG signal contains a plurality of human physiological and pathological information. Many clinical diseases, especially heart disease, can cause changes in the pulse. However, during an acquisition process, affected by the surrounding environment such as instruments, the acquired signal contains a plurality of noise interferences. A high-frequency noise such as a power line interference, an electromyographical (EMG) interference, etc. can make the PPG signal relatively fuzzy with a plurality of glitches. The disturbances may cause great problems in correctly determining changes in heart function. Therefore, it is necessary to develop methods and systems for detecting a noise in a PPG signal, such that a clean PPG signal may be obtained to facilitate a determination of signal quality during a PPG signal processing process, thereby further facilitating an identification and a processing of the PPG signal.

### SUMMARY

The present disclosure discloses a method. The method may include obtaining a vital sign signal, marking, using a first manner based on peak detection, a peak value and a position of the peak in the vital sign signal, analysing, based on a secondary peak value and a position of the secondary peak before the peak or a secondary peak value and a position of the secondary peak after the peak, a noise detection result of the first manner, obtaining a characteristic by performing a characteristic measurement on the vital sign signal using a second manner, wherein the second manner is different from the first manner, analysing a noise detection result of the second manner by comparing the characteristic with a predetermined threshold, and determining, based on the noise detection result of the first manner and the noise detection result of the second manner, a noise analysis result of the vital sign signal.

According to an embodiment of the present disclosure, the vital sign signal may include pulse wave information.

According to an embodiment of the present disclosure, the vital sign signal may include a photoplethysmography (PPG) signal.

According to an embodiment of the present disclosure, the first method may include: reading vital sign signal data in a window period, finding a maximum value in the window period and a position corresponding to the maximum value, wherein an amplitude of the maximum value is greater than the predetermined threshold.

According to an embodiment of the present disclosure, the window period may take at least 2 seconds.

According to an embodiment of the present disclosure, the second method may include at least one of a threshold crossing sample count (TCSC) algorithm, a time delay algorithm, and a kurtosis algorithm.

According to an embodiment of the present disclosure, the characteristic may be generated based on a binary character sequence constructed by a cosine window function and the vital sign signal.

According to an embodiment of the present disclosure, the characteristic may include a signal distribution density calculated based on a reconstructed trajectory of the vital sign signal.

According to an embodiment of the present disclosure, the characteristic may include a kurtosis calculation result.

According to an embodiment of the present disclosure, the analysing, based on a secondary peak value and a position of the secondary peak before the peak or a secondary peak value and a position of the secondary peak after the peak, a noise detection result of the first manner may include: determining a count of peak(s) in a current window period, a maximum peak and a minimum peak; when the count of peak(s) is greater than or equal to 2, and a difference between the maximum peak and the minimum peak is greater than a predetermined threshold, or the count of peak(s) is less than or equal to 1, determining that the vital sign signal of the current window period contains a noise.

The present disclosure also discloses a system including a storage executing a plurality of sets of instructions. The plurality of sets of instructions may be executed to detect a noise in a vital sign signal, and perform following operations: obtaining the vital sign signal, marking, using a first manner based on peak detection, a peak value and a position of the peak in the vital sign signal, analysing, based on a secondary peak value and a position of the secondary peak before the peak or a secondary peak value and a position of the secondary peak after the peak, a noise detection result of the first manner, obtaining a characteristic by performing a characteristic measurement on the vital sign signal using a second manner, wherein the second manner is different from the first manner, analysing a noise detection result of the second manner by comparing the characteristic with a predetermined threshold, and determining, based on the noise detection result of the first manner and the noise detection result of the second manner, a noise analysis result of the vital sign signal.

### BRIEF DESCRIPTION OF THE DRAWINGS

FIG. 1 is a schematic diagram illustrating an application scenario of a vital sign signal analysis system in the present disclosure;

FIG. 2 is a schematic diagram illustrating a vital sign signal analysis system in the present disclosure;

FIG. 3 is a flowchart illustrating an exemplary operation process of a system;

FIG. 4 is a schematic diagram illustrating an analysis module;

FIG. 5 is a flowchart illustrating an exemplary operation process of an analysis module;

FIG. 6 is a flowchart illustrating a process of an A algorithm in a vital sign signal analysis method in the present disclosure;

FIG. 7 is a flowchart illustrating a process of a B algorithm in a vital sign signal analysis method in the present disclosure.

FIG. 8 is an instance diagram illustrating a vital sign signal processed using a threshold crossing sample count (TCSC) algorithm.

FIG. 9 is a schematic diagram illustrating a distribution of data points of a photoplethysmography (PPG) signal in a phase space diagram.

### DETAILED DESCRIPTION

A vital sign signal analysis system provided in the present disclosure may be applied to a plurality of fields, including but not limited to: guardianship (including but not limited to elderly guardianship, middle-aged guardianship, youth guardianship, child guardianship, etc.), a medical diagnosis (including but not limited to an electrocardiogram diagnosis, a pulse diagnosis, a blood pressure diagnosis, a blood oxygen diagnosis, etc.), an exercise monitoring (including but not limited to a long-distance running monitoring, a middle and short-distance running monitoring, a sprinting monitoring, a cycling monitoring, a rowing monitoring, an archery monitoring, a horseback riding monitoring, a swimming monitoring, a climbing monitoring, etc.), a hospital care (including but not limited to a critical patient monitoring, a genetic disease patient monitoring, an emergency patient monitoring, etc.), a pet care (a critical pet care, a newborn pet care, a home pet care, etc.), or the like.

The vital sign signal analysis system may acquire one or more vital sign signals from living bodies, such as physical and chemical information of ECGs, pulses, blood pressures, blood oxygens, heart rates, body temperatures, HRVs, BPVs, brain waves, ultra-low frequency radio waves emitted by human bodies, breathing, musculoskeletal status, blood sugars, blood lipids, blood concentrations, platelet content, heights, weights, or the like. The vital sign signal analysis system may include a storage executing a plurality of sets of instructions. The plurality of sets of instructions may be executed to detect a noise in a vital sign signal, and may be executed further to obtain the vital sign signal, mark, using a first manner based on peak detection, a peak value and a position of the peak in the vital sign signal, analyse, based on a secondary peak value and a position of the secondary peak before the peak or a secondary peak value and a position of the secondary peak after the peak, a noise detection result of the first manner, obtain a characteristic by performing a characteristic measurement on the vital sign signal using a second manner, wherein the second manner is different from the first manner, analyse a noise detection result of the second manner by comparing the characteristic with a predetermined threshold, and determining, based on the noise detection result of the first manner and the noise detection result of the second manner, a noise analysis result of the vital sign signal. An output module may be configured to output an analysis result. The analysis system may effectively detect a noise in vital sign signal data by using a small amount of calculation, and make corresponding matching and marking. The system may be easily applied to portable devices or wearable devices. The system may continuously monitor vital sign signals of living bodies in real time (or non-real time), and transmit monitoring results to external devices (including but not limited to storage devices or cloud servers). For example, the system may continuously monitor vital sign signals of a user in a random period of time, such as minutes, hours, days, or months, or continuously monitor vital sign signals of the user at regular intervals. The system may display vital sign signal status of monitored living bodies in real time (or non-real time), such as information of a pulse, a blood pressure, a blood oxygen concentration, etc., and provide physiological information data to relevant remote third parties, such as hospitals, nursing institutions, related persons, or the like. For example, a user may use the system at home. Vital sign signal status or physiological information data of the user monitored by the system may be provided to remote hospitals, nursing institutions, related persons, or the like. Part or all of the vital sign signal status or the physiological information data of the user may also be stored in a local or remote storage device. The physiological information data may be transmitted wired or wirelessly. A noise in the vital sign signal acquired may be effectively detected, and may be matched and marked correspondingly (such that the system may be easily applied to portable devices or wearable devices). In particular, the analysis system may continuously monitor the vital sign signals of living bodies in real time (or non-real time), and transmit monitoring results to external devices (including but not limited to storage devices or cloud servers). The analysis system may output and display vital sign signal status of monitored living bodies in real time (or non-real time), such as an ECG, a pulse, a blood pressure, a blood oxygen concentration, etc., and provide the vital sign signals to related third parties, such as hospitals, nursing structures, or related persons, or the like. All transmission processes related to vital sign signals described above may be wired or wireless.

The above descriptions of application fields are merely provided as specific examples and should not be regarded as the only feasible implementation. Obviously, after understanding basic principles of the methods and systems for analysing vital sign signals, those skilled in the art may make various modifications and variations in forms and details of application fields for implementing the above methods and systems without departing from the principles. However, those modifications and variations may be still within the scope of the above descriptions.

In order to illustrate the technical solutions in the embodiments of the present disclosure, a brief introduction of the drawings referred to in the description of the embodiments is provided below. Obviously, drawings described below are only embodiments of the present disclosure. For those skilled in the art, the present disclosure may be applied to other similar scenarios according to the drawings without creative efforts. Unless apparent from the locale or otherwise stated, like reference numerals represent similar structures or operations throughout the several views of the drawings.

As used in the disclosure and the appended claims, the singular forms “a,” “an,” and/or “the” may include plural forms unless the content clearly dictates otherwise. In general, the terms “comprise,” “comprises,” and/or “comprising,” “include,” “includes,” and/or “including,” merely prompt to include steps and elements that have been clearly identified, and these steps and elements do not constitute an exclusive listing. The methods or devices may also include other steps or elements.

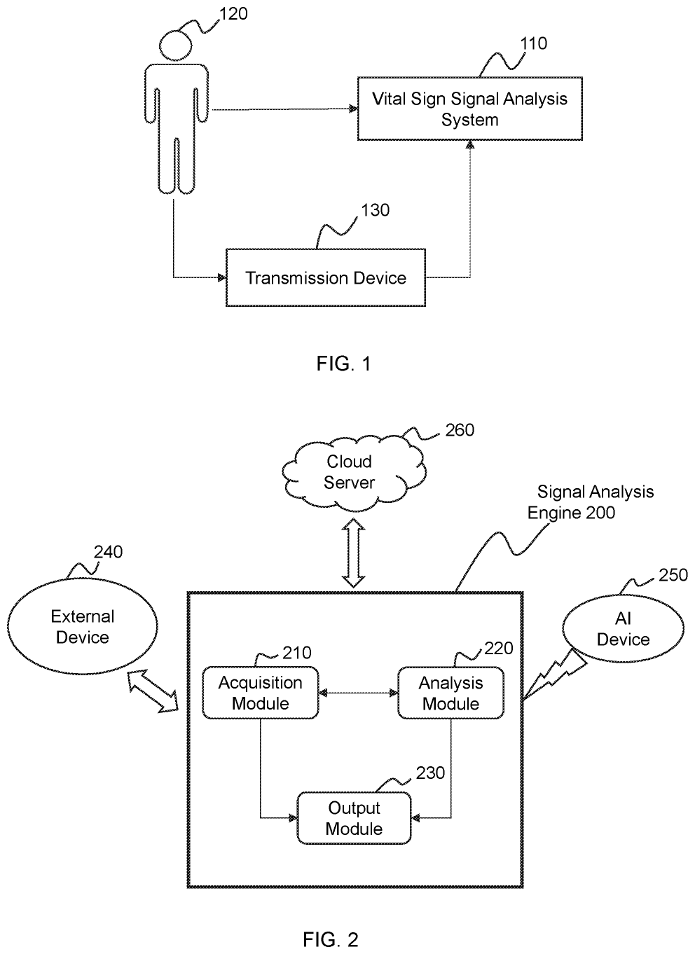

FIG. 1 is a schematic diagram illustrating an application scenario of a vital sign signal analysis system in the present disclosure. The application scenarios may include but is not limited to a vital sign signal analysis system 110, a living body 120 and a transmission device 130. The vital sign signal analysis system 110 may be configured to extract, receive, acquire, analyse, and/or process vital sign signals from the living body 120. The living body 120 may include but is not limited to human bodies, and is not limited to a single living body. The vital sign signals may include but not limited to physical and chemical information of ECGs, pulses, blood pressures, blood oxygens, heart rates, body temperatures, HRVs, BPVs, brain waves, ultra-low frequency radio waves emitted by human bodies, breathing, musculoskeletal status, blood sugars, blood lipids, blood concentrations, platelet content, heights, weights, or the like. The transmission device 130 may include but is not limited to processors, sensors, embedded devices based on single chip computers, ARM, etc., and electronic, mechanical, physical, and chemical devices such as analysers, detectors etc. Transmission methods may include but are not limited to wired or wireless methods such as a radar, an infrared, a Bluetooth, a wire, an optical fiber, etc. Information transmitted may be analog or digital, or may be real-time or non-real-time. The device may be aimed at a specific living body, and may also be aimed at a certain group, a type or a plurality of types of living bodies. The device may also include a central database or a cloud server. The vital sign signal analysis system 110 may obtain vital sign signals directly or indirectly. The vital sign signals acquired may be transmitted directly to the vital sign signal analysis system 110, or may be transmitted to the vital sign signal analysis system 110 via the transmission device 130. Devices used for acquiring vital sign signals may include but are not limited to a heartbeat collection device, an electrocardiogram detector, a pulse wave detector, a brain wave detector, a blood pressure measuring instrument, a vital sign signal detection device, a human breath detector, or the like. The vital sign signals may also be acquired using smart wearable devices having functions of the above-mentioned devices, such as watches, earphones, glasses, accessories, portable devices, or the like. In some embodiments, vital sign signals of human bodies may also be acquired using smart cloth disposed with sensors (e.g., photoelectric sensors or pressure sensors).

The above description of application scenarios of the vital sign signal analysis system is merely provided as a certain specific example, and should not be regarded as the only feasible implementation. Obviously, after understanding basic principles of the vital sign signal analysis system, those skilled in the art may make various modifications and variations in forms and details of application methods of the vital sign signal analysis system without departing from the principles. However, those modifications and variations are still within the scope of the above descriptions. For example, information acquired from the living body 120 may be directly transmitted to the vital sign signal analysis system 110 without passing through the transmission device 130. The vital sign signal analysis system 110 may also obtain a plurality of types of vital sign signals from a plurality of living bodies 120 directly for comprehensive processing at the same time. Those modifications and variations may be still within the protection scope of the claims in the present disclosure.

FIG. 2 illustrates a schematic diagram of a vital sign signal analysis system, including but not limited to one or more signal analysis engines 200, one or more external devices 240, one or more artificial intelligence (AI) devices 250, and a cloud server 260, or the like. The signal analysis engine 200 may include but is not limited to an acquisition module 210, an analysis module 220 and an output module 230. The acquisition module 210 may be mainly configured to acquire vital sign signals in the vital sign signal analysis system. The module may be implemented by photoelectric sensing or electrode sensing. The module may obtain vital sign signals based on a temperature sensing, a humidity change, a pressure change, a photoelectric induction, a body surface potential change, a voltage change, a current change or a magnetic field change, etc. The acquisition module may obtain various information of acoustics, optics, magnetism, heat, or the like. Types of the information may include but are not limited to vital sign signals such as pulse information, heart rate information, electrocardiogram information, blood pressure information, blood oxygen information, breathing information, or the like. For example, the acquisition module may obtain information related to pulse wave including but not limited to waveform information such as waveform, time interval, peak, trough, amplitude, or the like. The acquisition module 210 may make full use of various devices such as a local pulse wave acquisition device, a remote wireless remote pulse wave monitoring system, a medical pulse wave monitoring system or a portable pulse wave monitoring device for home use, a traditional pulse wave monitoring device, or a portable smart wearable device having a pulse wave monitoring function, such as a watch, an pair of earphone, or the like. According to needs, the acquisition module 210 may acquire complete vital sign signals as needed, or acquire vital sign signals within a certain time interval, such as a window period within 2 seconds (2 s).

A certain calibration module may be integrated inside the acquisition module 210, or a separate calibration module (not shown in the figure) may be disposed in the signal analysis engine 200, which may be configured to adjust, optimize, calibrate or remove unrelated error interference on the acquired vital sign signals. An acquisition of vital sign signals may be affected by many factors which may affect characteristics of the vital sign signals, such as a waveform, a peak amplitude, a peak point interval, or the like. For example, vital sign signals of one living body may be different at different times during one day. Vital sign signals of one living body may be different in different life states, such as an exercise state or a resting state, a load working state or a sleep state, a happy state or an irritable state, or the like. Vital sign signal of one living body taking or not taking medicines may also be different. In addition, vital sign signals of different living bodies in a same state may be different. Therefore, a corresponding calibration module may be integrated in the acquisition module 210, or a corresponding calibration module (not shown in the figure) may be disposed in the signal analysis engine 200 to adjust, optimize, calibrate or remove the error interference so as to obtain accurate vital sign signals. In addition, the acquisition module 210 may adjust parameters for different living bodies, and store vital sign signals acquired from one living body in the cloud server 260, so that the acquisition module 210 may have an adaptive function to form a library of individual vital sign signals of one living body, thereby making the acquired vital sign signals more accurate. In addition, the photoelectric sensing may be affected by factors such as a light intensity, a skin color, a skin roughness, a skin temperature, a skin humidity, an ambient temperature, an ambient humidity, or the like. Therefore, a corresponding environmental adaptation module, such as a correction or compensation module corresponding to environmental factors may need to be the integrated in the acquisition module 210. The modifications, changes or variations of the vital sign signal analysis system should be within the protection scope of the present disclosure.

The analysis module 220 may mainly be configured to calculate, analyse, determine, and/or process vital sign signals. The analysis module 220 may be centralized, distributed, local, or remote. A calculation method may be a specific calculation, or a yes/no analysis based on a threshold. An analysis process may be real-time or non-real-time. A calculation process may be performed directly by the system or by an external computer program. A device used in the calculation process may be an internal device of the system or an external device of the system. A processing process may be real-time or non-real-time, and may be executed directly by the system or by a connected external device. The output module 230 may be configured to output calculated, analysed, determined, and/or processed vital sign signals. Output information may be analog or digital, and may be logical analysis result of yes/no or may be processed vital sign signals. An output process may be real-time or non-real-time, and may be executed directly by the system or by a connected external device. The external device 240 may refer to a variety of direct or indirect devices related to a module of the vital sign signal analysis system. The device may be local or remote, and may be wired or wireless. For example, the external device 240 may be an LED or LCD screen configured to display vital sign signals, or storage devices such as a hard disk, a floppy disk, etc. configured to store vital sign signals. The AI device 250 may generally refer to a hardware or a software with a self-learning function based on data, including but not limited to various types of central processing units (CPU), graphics processing units (GPU), tensor processing units (TPU), ASIC, and various software and hardware devices executing support vector machines (SVM), Logistic regressions (LR), long-range short-term memory models (LSTM), generated adversarial networks (GAN), Monte Carlo tree searches (MCTS), hidden Markov models (HMM), Random forests, Recursive Cortical Networks (RCN), or the like.

The cloud server 260 may be configured to store all data related to an operation of the vital sign signal analysis system, and may provide data call support for each module in the system in real time or non-real time. The cloud server 260 may be configured as a cloud database of the vital sign signal analysis system.

The analysis module 220 may be connected to the acquisition module 210 wired or wirelessly. The acquisition module 210 and analysis module 220 may be connected to the output module 230 wired or wirelessly. The acquisition module 210, the analysis module 220 and the output module 230 may be connected to different power supplies, or share two or three power supplies. The acquisition module 210, the analysis module 220 and the output module 230 may be connected to an external device respectively. The external device may be connected to one or more modules wired or wirelessly. The signal analysis engine 200 may be connected to the cloud server 260 wired or wirelessly. The modules and devices described above may be not necessary. After understanding content and principles of the present disclosure, those skilled in the art may make various modifications and variations in forms and details of the system without departing from the principles and structures. Each module may be arbitrarily combined, and some modules may be added or deleted as needed. However, those modifications and variations may be still within the protection scope of the claims in the present disclosure. For example, the acquisition module 210 and the output module 230 in FIG. 2 may be integrated into a single module, which may have functions of information acquisition and information output. The module may be connected to the analysis module 220 wired or wirelessly. Corresponding storage devices may be integrated inside each module for short-term cache of information data during system execution, or for long-term storage of the information data. A corresponding independent storage module may also be added in the signal analysis engine 200 so as to store obtained, and/or calculated, analysed, and processed vital sign signals. These modifications and variations may be still within the scope of protection of the claims of the present disclosure.

Connections between each module in the vital sign signal analysis system, connections between modules and external devices, and connections between the system and storage devices or cloud servers are not limited to the above description. The connection methods above may be used singly, or in combination in the analysis system. Various modules may also be integrated together in order to achieve functions of one or more modules through one device. External devices may also be integrated in implementation devices of one or more modules, and a single or a plurality of modules may also be integrated in a single or a plurality of external devices. Each module, modules and external devices, and the system and storage devices or cloud servers in the vital sign signal analysis system may be connected wired or wirelessly. Wired connections may include but are not limited to wire connection ways such as wires, optical fiber, or the like. Wireless connections may include but are not limited to wireless connection ways such as various radio communications including Bluetooth, infrared, or the like.

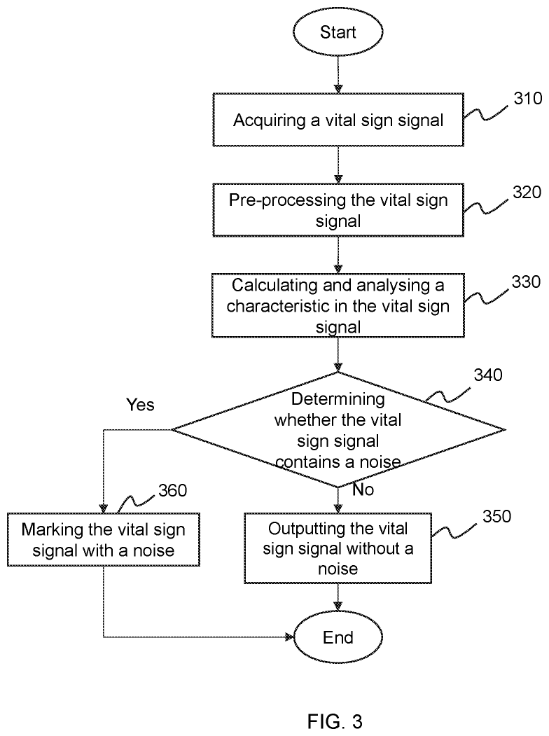

FIG. 3 is a flowchart illustrating an exemplary operation process of the vital sign signal analysis system. The process may include following operations. In 310, a vital sign signal may be acquired. The vital sign signal data may be stored in the acquisition module 210 in FIG. 2, or in corresponding storage devices (not shown in the figure), or in the cloud server 260, or may be directly used in a next operation without being stored. In 320, the vital sign signal data may be pre-processed. The pre-processing operation may be performed by the analysis module 220 or other separate pre-processing modules (not shown in the figure). An effect of information optimization may be achieved via a pre-processing of information data. A pre-processing manner may include but is not limited to correcting, changing or removing part of noise information or redundant information in the information data. Specific processing manners may include but are not limited to a low-pass filtering, a band-pass filtering, a wavelet transform filtering, a median filtering method, a morphological filtering, a curve fitting method, or the like. After the preprocessing operation, a part of clearly identifiable noise, such as a baseline drift noise may be removed from the vital sign signal data. After the pre-processing, in 330, a characteristic of the vital sign signal may be calculated and analysed. The operation may be performed by the analysis module 220. One or more algorithms in the analysis module 220 may calculate and analyse the characteristic of the vital sign signal. After a calculation and an analysis, in 340, whether there is a noise in the vital sign signal may be determined. If an calculation result is that there is no noise, in 350, the vital sign signal without a noise may be outputted by the output module 230. If the calculation result is that there is a noise in the vital sign signal, in 360, the output module 230 may mark the vital sign signal with a noise and output marked vital sign signal.

The methods and operations described herein may be implemented in any suitable order under appropriate circumstances or may be implemented simultaneously. In addition, individual operations may be deleted from any one method without departing from the spirit and scope of the subject matter described herein. Aspects of any example described above may be combined with aspects of any of the other examples described so as to constitute further examples without losing desired effects. For example, the pre-processing operation 320 may be unnecessary, or other selection conditions may be added between the pre-processing operation and the analysis processing operation, such as storing and backuping results of the pre-processing or results generated by any operation in the processing.

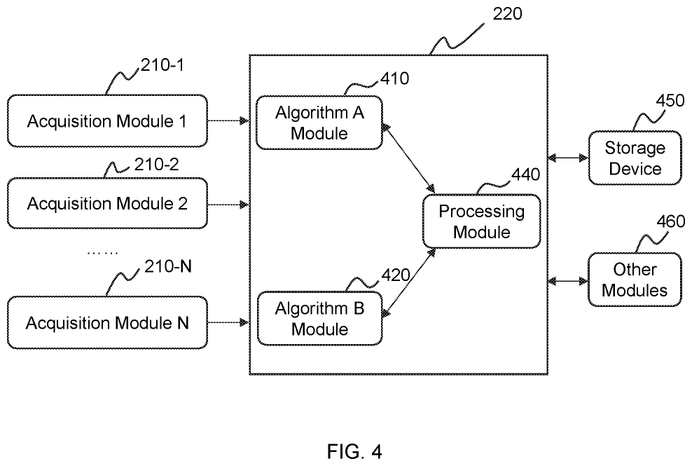

FIG. 4 is a schematic diagram illustrating the analysis module 220 and surrounding devices. The analysis module 220 may include an A algorithm module 410, a B algorithm module 420, and a processing module 440. The analysis module 220 may be connected to a storage device 450 and other modules 460. The storage device 450 may be integrated in the analysis module 220, or may be integrated in the acquisition module 210, or may be an independent storage device. The analysis module 220 may be selectively connected with other one or more acquisition modules 210-1, 210-2, and 210-N. All modules or devices mentioned here may be connected wired or wirelessly. Three algorithm modules 410, 420 and the processing module 440 within the analysis module 220 may be connected in pairs or may be connected to other modules separately. Connection between the modules may be not limited to that shown in FIG. 4. The above description of the analysis processing module is merely provided as a specific example and should not be considered as the only feasible implementation. Each module may be realized by one or more components, and functions of each module may be not limited herein. After understanding basic principles of the analysis and process, those skilled in the art may make various modifications and variations in forms and details of specific implementations and operations of the analysis and processing module, and may also make some simple deductions or replacements without departing from the principles, and may make certain adjustments or combinations to the order of the modules without making creative efforts. However, those modifications and variations may be still within the scope of the above descriptions. For example, the analysis module 220 may perform different functions, or simply determine whether there is a noise in a vital sign signal, or perform a denoising process on the vital sign signal. When the analysis module 220 only performs a noise analysis function, the processing module 440 may be unnecessary. Similarly, the two algorithm modules in the analysis module 220 may co-exist or may exist separately. When the analysis module 220 is running, one or more modules of algorithm modules may be selectively run, or modules may be run successfully in stages or simultaneously, or the algorithm modules may be run at other time combinations. Further, any one of the algorithm modules may perform calculation processing on results of other one or more algorithm modules, or transmit results generated by different algorithm modules to processing modules simultaneously or non-simultaneously for processing.

All vital sign signal data may be selectively stored in the storage device 450 after being received, calculated, analysed, determined, and/or processed by the analysis module 220 so as to be read and analysed by the analysis module 220 at any time in any subsequent operation. The storage devices 450 mentioned here may generally refer to all media that can read and/or write information, such as but not limited to a random access memory (RAM) and a read-only memory (ROM). For example, the storage devices 450 may include various storage components such as a hard disk, a floppy disk, a USB disk, an optical disks, or the like. The RAM may include but is not limited to a dekatron, a selectron tube, a delay line memory, a Williams tube, a dynamic random access memory (DRAM), a static random access memory (SRAM), a thyristor random access memory (T-RAM), a zero capacitor random access memory (Z-RAM), or the like. The ROM may include but is not limited to a bubble memory, a twistor memory, a film memory, a plated wire memory, a magnetic-core memory, a drum memory, a CD-ROM, a hard disk, a tape, a non-volatile random access memory (NVRAM), a phase-change memory, a magneto-resistive random access memory, a ferroelectric random access memory, a non-volatile SRAM, a flash memory, an electrically erasable programmable read-only memory, an erasable programmable read-only memory, a programmable read-only memory, a mask ROM, a floating gate random access memory, a Nano random access memory, a racetrack memory, a resistive random access memory, a programmable metallization unit, or the like. The storage devices mentioned above are merely provided as examples. The system may use a storage device that is not limited in the present disclosure.

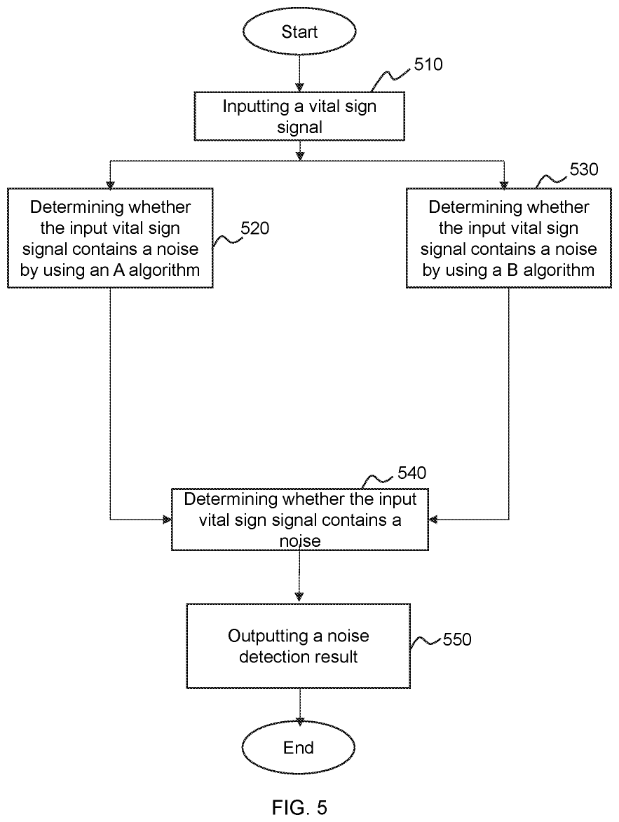

FIG. 5 is a flowchart illustrating a process of calculation, analysis, determination, and processing performed by the analysis module 220 on a vital sign signal. In 510, a vital sign signal in a living body may be input and read first. Fruther, algorithm calculation operations 520 and 530 may be performed after information is read. The algorithm calculation operations may be unnecessary. One or more of the algorithm calculation operations may be selected, or may be executed independently, or in a certain order, or simultaneously. Taking a process of first executing an A algorithm as an example, an calculation process of the A algorithm may be performed in operation 520, and a calculation result may be transmitted to operation 540 for a comprehensive analysis. Taking a process of executing a B algorithm as an another example, a calculation process of the B algorithm may be performed in operation 530. In 530, a noise in the vital sign signal may be calculated and analysed, and a calculation and analysis result may be transmitted to the analysis operation 540. After whether the current vital sign signal contains a noise is determined in the analysis operation 540, a noise reporting operation 550 may be executed, which may output a noise detection result.

In the same way, a B algorithm calculation operation 530 may be performed after information is read. A calculations result may be transmitted to a determination operation 540. After whether the current vital sign signal contains a noise is determined in the determination operation 540, a noise reporting operation 550 may be executed, which may output a noise detection result. If the calculation result shows that there is a noise in the current information, the current noise result may be output by the output module 230, and the analysis process may be ended, or the result that the current information contains a noise may be transmitted to a noise processing operation 560 (not shown in the figure), in which the noise identified in the information may be removed, and the analysis process may be ended.

The above description of the vital sign signal analysis process is merely provided as a specific example, and should not be regarded as the only feasible implementation. Obviously, after understanding basic principles of algorithms, those skilled in the art may make various modifications and variations in forms and details of specific implementations and steps of information analysis and processing without departing from the principles. However, those modifications and variations may be still within the scope of the above descriptions. For example, data generated during a calculation using the A algorithm may be processed during a calculation using the B algorithm, or data generated during a calculation using the B algorithm may be processed during a calculation using the A algorithm, or calculation results between the A algorithm and the B algorithm may be recycled.

The A algorithm and B algorithm described above may calculate different characteristics in the read information, and may also calculate and analyse a same characteristic in the read information in different ways. Operations 520 and 530 in FIG. 5 may be interchanged, and the two algorithms may also be executed in a freely combined order. For example, a calculation may be performed on the information read using the B algorithm. Whether a calculation result contains a noise may be further determined. The calculation result that contains a noise may be transmitted to the A algorithm operation for further calculation and analysis. In a specific embodiment, in a process using the A algorithm, a vital sign signal may be calculated and analysed according to waveform distribution of acquired information. Whether the vital sign signal contains a noise may be determined according to the calculation result. If the analysis result is that the current information does not contain a noise, the calculation and analysis process may be ended, and the output module 230 may output a noise analysis result. If the analysis result is that the current information contains a noise, a further noise recognition process may be performed using the B algorithm, or the noise analysis result 230 may directly output a noise analysis result. In a process using the B algorithm, a characteristic of a vital sign signal may be obtained in calculation. Whether the information contains a noise may be determined based on a predetermined characteristic threshold. For example, according to the B algorithm, several characteristics may be obtained using a threshold crossing sample count (TCSC) algorithm, a time delay algorithm (TDA), and a kurtosis calculation. A noise may be analysed and determined by determining thresholds of the characteristics. After a calculation and analysis using the algorithm, a noise analysis result may be outputted by the output module 230.

The above may be a brief introduction to characteristics of the two algorithms, and may not represent the only feasible implementation. After understanding relevant algorithm principles, those skilled in the art may expand or develop the three algorithms may be to different degrees, or increase or decrease the steps, or effectively arrange and combine the various steps, so as to achieve better calculation and analysis results.

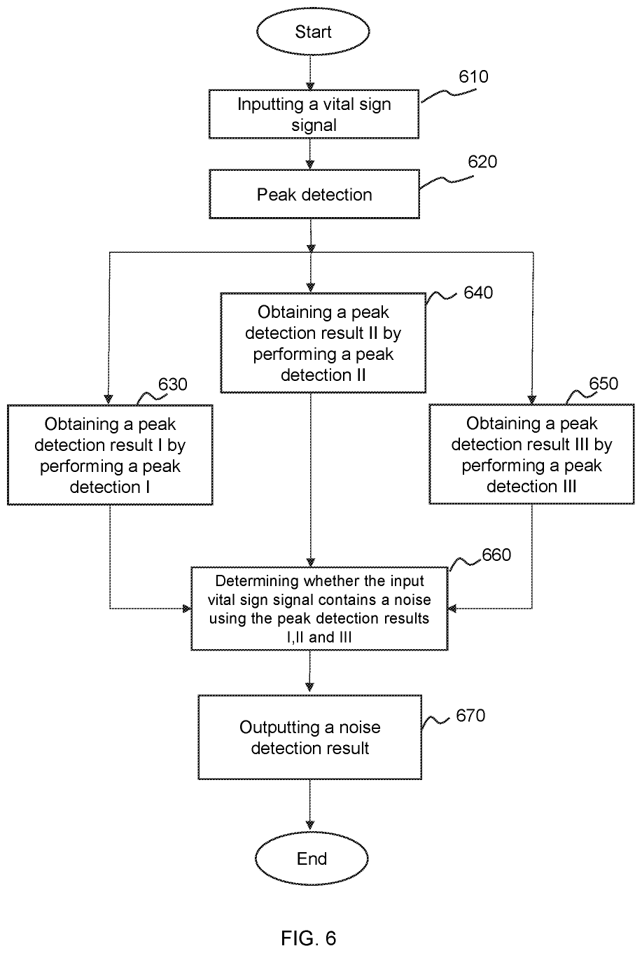

FIG. 6 is a flowchart illustrating a calculation and analysis process performed using an A algorithm. In 610, a vital sign signal in a window period (marked as L_s) may be first input. The vital sign signal may be a pulse wave signal. The pulse wave signal may be obtained by using a photoelectric volume pulse wave measurement method, or may be a pressure wave signal obtained using a pressure sensor. The window period of the vital sign signal may be related to physiological characteristics of a relevant individual. In some embodiments, the window period may be related to a heart rate of a relevant individual. For example, when the heart rate of the relevant individual increases, the window period may be shorter. In some embodiments, the window period may be set to be 2 seconds (2 s).

In 620, peak detection may be performed on the vital sign signal in the window using a peak detection algorithm. Specifically, the operation may include following sub-steps:

In Step 1, vital sign signal data in a window period (for example, a data window of 2 s) may be read, a maximum value in the window period and a position corresponding to the maximum value may be searched. An amplitude of the maximum value herein may be greater than a predetermined threshold;

In Step 2, a certain data window with the position corresponding to the maximum value as a center may be selected, and a maximum value in the data window and a position corresponding to the maximum value may be searched;

In Step 3, repeated maximum value(s) may be deleted, a maximum value may be retained as a peak, and a position corresponding to the maximum value may be retaied as a peak position.

Further, in 630, a peak detection result I may be obtained by performing a peak detection I. Specifically, a count of peak(s) in a current window period, a maximum peak and a minimum peak may be determined based on the peak detection algorithm. When the count of peak(s) is greater than or equal to 2, and a difference between the maximum peak and the minimum peak is greater than a predetermined threshold, or the count of peak(s) is less than or equal to 1, the PPG signal in the current window period may be determined to contain a noise.

In 640, a peak detection result II may be obtained by performing a peak detection II. Specifically, local data points before the peak may be analysed, and data within 0.12 seconds before the peak may be determined as local data before the peak. A secondary peak not higher than the peak in the local data may be searched. If there is a secondary peak, the PPG signal in the current window period may be determined to contain a noise.

In 650, a peak detection result III may be obtained by performing a peak detection III. Specifically, a local data point after the peak may be analysed, and data within 0.16 seconds after the peak may be determined as local data after the peak. A secondary peak not higher than the peak in the local data may be searched. If there is a secondary peak, a valley between the peak and the secondary peak may be searched. If an amplitude of the valley and the secondary peak is greater than a predetermined threshold, the PPG signal in the current window period may be determined to contain a noise.

In 660, whether an input vital sign signal contains a noise may be determined by using peak detection results I, II and III. In some embodiments, if the input vital sign signal contains a noise in any one of the peak detection results I, II and III, the input vital sign signal may be determined to contain a noise. Finally, in 670, a noise detection result of the A algorithm may be outputted.

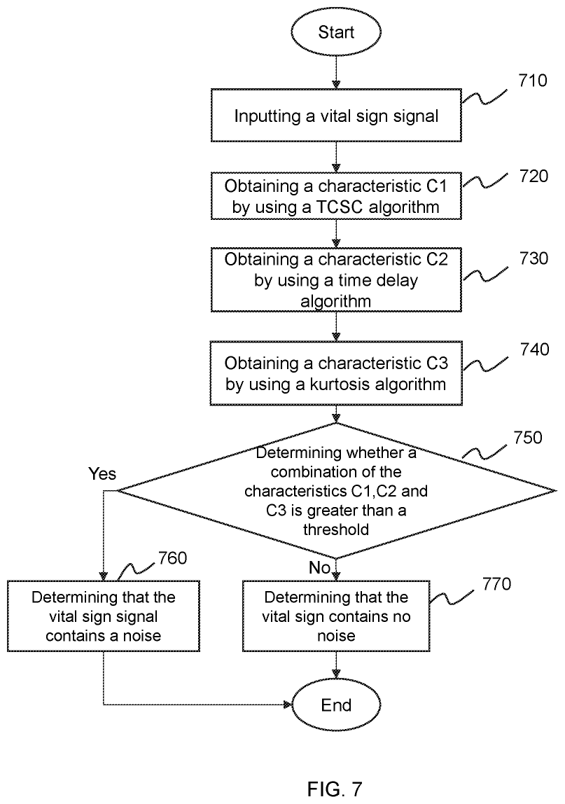

FIG. 7 is a flowchart illustrating a calculation and analysis process performed using a B algorithm. In 710, a vital sign signal in a window period (marked as L_s) may be input first. The vital sign signal may be a pulse wave signal. The pulse wave signal may be obtained using a photoelectric volume pulse wave measurement method, or may be a pressure wave signal obtained using a pressure sensor. The window period of the input vital sign signal may be related to physiological characteristics of a relevant individual. In some embodiments, the window period may be related to a heart rate of a relevant individual. For example, when the heart rate of the related individual increases, the window period may be shorter. In some embodiments, the window period may be set to be 2 seconds (2 s).

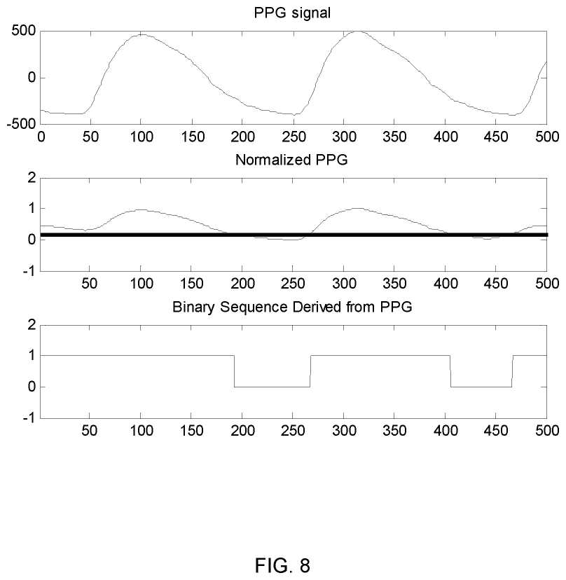

In 720, a characteristic C1 may be obtained by processing the vital sign signal using a threshold crossing sample count (TCSC) algorithm. Changes of a PPG signal processed by using the TCSC algorithm may be illustrated in FIG. 8. Specifically, vital sign signal data of a current window period may be first multiplied by a cosine window function φ(t). An equation of the window function may be:

$$ϕ ( t ) = \{ \begin{matrix}\frac{1}{2} ( 1 - cos ( 4 π t ) ) & 0 \leq t \leq \frac{1}{4} \\ 1 & \frac{1}{4} \leq t \leq {L}_{s} - \frac{1}{4} \\ \frac{1}{2} ( 1 - cos ( 4 π t ) ) & {L}_{s} - \frac{1}{4} \leq t \leq {L}_{s}\end{matrix}$$

where Lₛ in the equation denotes a time length of the window, which may take 2 s. Further, newly obtained data may be normalized.

Further, each normalized sampling point xᵢ(i=1, 2, . . . , n, wherein n denotes a count of sampling point(s)) may be converted into a 0-1 string bi, i=1, 2, . . . n after being compared with a threshold V₀. The threshold V₀ may be selected from a threshold interval. In some embodiments, the threshold interval may be [0.1, 0.4]. In some further embodiments, the threshold interval may be [0.2, 0.3]. If xᵢ>V₀, a corresponding string bᵢ=1, otherwise, bᵢ=0.

A count of 1 in a binary sequence b={b₁b₂ . . . bₙ} may be determined, and then N may be calculated. N may be calculated using an equation:

$$N = \frac{{n}_{0}}{n} \times 100$$

where n₀ denotes a count of sampling point(s) greater than a threshold V₀, n denotes a count of sampling point(s).

Finally, the characteristic C1 may be determined as a function of N. In some embodiments, C1 may be determined as a piecewise constant function of N. For example, if N>80 or N<90, C1 may be a constant, such as 1. If N is greater than or equal to 90, C1 may be 2. In other cases, C1 may be 0.

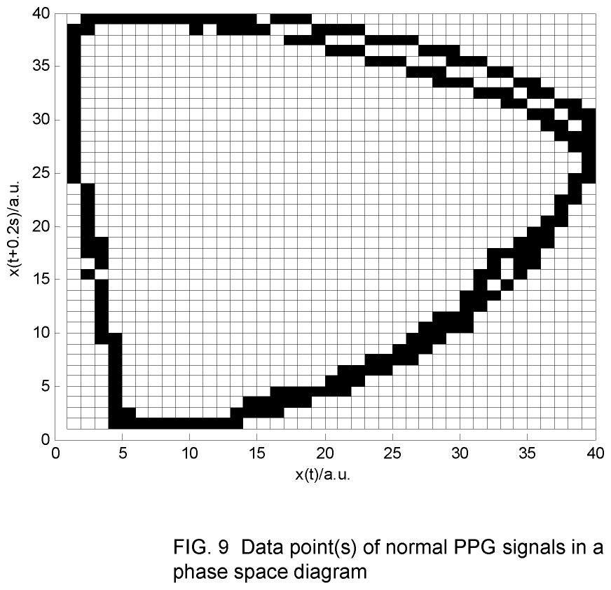

In 730, a characteristic C2 may be obtained by processing the vital sign signal using a time delay algorithm (TDA). TDA may be a algorithm based on a phase space reconstruction. Specifically, a vital sign signal x(t) may be drawn in a figure in the following way: x axis abscissa denotes x(t) and y axis ordinate denotes x(t+τ), wherein τ is a preset time constant. A graph drawn in this way may be referred to as a two-dimensional phase space graph. As shown in FIG. 9, a two-dimensional graph may be first covered with a 40×40 square grid, and then a distribution density d of a signal reconstructed trajectory may be calculated. A equation may be expressed as:

$$d = \frac{count of grids covering signal}{count of all grids ( 1600 )}$$

If d>d0, wherein d0 denotes a characteristic density, C2 may be 1, otherwise, C2 may be 0. In some embodiments, d0 may be a constant not greater than 0.181.

In 740, a characteristic C3 may be obtained by processing the vital sign signal using a kurtosis algorithm. The Kurtosis calculation (i.e., Kurtosis) is a statistic describing steepness of distribution of all values of a variable. Specifically, an equation of the kurtosis algorithm may be:

$$K = \frac{1}{N} {\sum }_{i = 1}^{N} {[ \frac{{x}_{i} - \overline{x}}{s} ]}^{4}$$

where x̅ denotes a mean of data points, s denotes a variance of the data points, and N denotes a count of the data point(s). In general, the kurtosis may reflect sharpness of a peak. A kurtosis of a normal distribution may be 3. If K<3, a distribution may have an insufficient kurtosis. If K>3, the distribution may have an excessive kurtosis.

Finally, a characteristic C3 may be determined as a function of K. In some embodiments, C3 may be set as a piecewise constant function of K. For example, if K>2.95, C3 may be a constant, such as 3.

In 750, a combination based on the characteristics C1, C2 and C3 may be calculated and compared with a statistical noise threshold V1. In some embodiments, the combination based on C1, C2, and C3 may be a linear combination of C1, C2, and C3. For example, the combination may be C1+C2+C3. The statistical noise threshold V1 may be within a value interval. In some embodiments, the value interval of V1 may be [1, 3].

If the combination based on the characteristics C1, C2 and C3 is greater than the statistical noise threshold V1, in 760, the input vital sign signal may be determined to contain a noise. If the combination based on the characteristic C1, C2 and C3 is not greater than the statistical noise threshold V1, in 770, the input vital sign signal may be determined to contain no noise. At this point, the process of the entire B algorithm may be ended.

The above descriptions of the calculation process of the B algorithm are merely provided as specific examples, and should not be regarded as the only feasible implementation. Obviously, after understanding basic principles of kurtosis calculation and noise analysis, those skilled in the art may make various modifications and variations in forms and details of specific implementations and steps of the B algorithm without departing from the principles. However, those modifications and variations may be still within the scope of the above descriptions. For example, during an execution process of B algorithm, operation 730 may be skipped and the kurtosis calculation may be directly performed in operation 740. The kurtosis calculation in operation 740 may be specifically performed in various forms, such as a direct calculation, a simulation, or the like. After the execution process of B algorithm, the calculation and analysis process may be ended, or a further calculation and analysis may be performed by an A algorithm module. Similarly, the A algorithm calculation process may also be performed simultaneously with the B algorithm calculation process.

The above-mentioned embodiments are only specific implementations of the present disclosure. The embodiments are described specifically and in detail but are not intended to be limiting. It should be noted that those skilled in the art, without departing from concept of the present disclosure, may make several variations and improvements, such as new characteristics or any new combinations disclosed in the present disclosure, as well as steps of the new manners or any new combination disclosed, which may be within the protection scope of the present disclosure.

We claim:

1. A method for detecting a noise in a vital sign signal, comprising:
obtaining the vital sign signal;
marking, using a first manner based on peak detection, a peak value and a position of the peak in the vital sign signal;
analysing, based on a secondary peak value and a position of the secondary peak before the peak or a secondary peak value and a position of the secondary peak after the peak, a noise detection result of the first manner;
obtaining a characteristic by performing a characteristic measurement on the vital sign signal using a second manner, wherein the second manner is different from the first manner;
analysing a noise detection result of the second manner by comparing the characteristic with a predetermined threshold; and
determining, based on the noise detection result of the first manner and the noise detection result of the second manner, a noise analysis result of the vital sign signal.

2. The method of claim 1, wherein the vital sign signal includes pulse wave information.

3. The method of claim 2, wherein the vital sign signal includes a photoplethysmography (PPG) signal.

4. The method of claim 1, wherein the first manner includes: reading vital sign signal data in a window period, finding a maximum value in the window period and a position corresponding to the maximum value, wherein an amplitude of the maximum value is greater than the predetermined threshold.

5. The method of claim 4, wherein the window period takes at least 2 seconds.

6. The method of claim 1, wherein the second manner includes at least one of a threshold crossing sample count (TCSC) algorithm, a time delay algorithm, and a kurtosis algorithm.

7. The method of claim 1, wherein the characteristic is generated based on a binary character sequence constructed by a cosine window function and the vital sign signal.

8. The method of claim 1, wherein the characteristic includes a signal distribution density calculated based on a reconstructed trajectory of the vital sign signal.

9. The method of claim 1, wherein the characteristic includes a kurtosis calculation result.

10. The method of claim 1, the analysing, based on a secondary peak value and a position of the secondary peak before the peak or a secondary peak value and a position of the secondary peak after the peak, a noise detection result of the first manner includes: determining a count of peak(s) in a current window period, a maximum peak and a minimum peak; when the count of peak(s) is greater than or equal to 2, and a difference between the maximum peak and the minimum peak is greater than a predetermined threshold, or the count of peak(s) is less than or equal to 1, determining that the vital sign signal of the current window period contains a noise.

11. A system, comprising a storage executing a plurality of sets of instructions, wherein the plurality of sets of instructions are executed to detect a noise in a vital sign signal and perform following operations:
obtaining the vital sign signal;
marking, using a first manner based on peak detection, a peak value and a position of the peak in the vital sign signal;
analysing, based on a secondary peak value and a position of the secondary peak before the peak or a secondary peak value and a position of the secondary peak after the peak, a noise detection result of the first manner;
obtaining a characteristic by performing a characteristic measurement on the vital sign signal using a second manner, wherein the second manner is different from the first manner;
analysing a noise detection result of the second manner by comparing the characteristic with a predetermined threshold; and
determining, based on the noise detection result of the first manner and the noise detection result of the second manner, a noise analysis result of the vital sign signal.

12. The system of claim 11, wherein the vital sign signal includes pulse wave information.

13. The system of claim 12, wherein the vital sign signal includes a photoplethysmography (PPG) signal.

14. The system of claim 11, wherein the first manner includes: reading vital sign signal data in a window period, finding a maximum value in the window period and a position corresponding to the maximum value, wherein an amplitude of the maximum value is greater than the predetermined threshold.

15. The system of claim 14, wherein the window period takes at least 2 seconds.

16. The system of claim 11, wherein the second manner includes at least one of a threshold crossing sample count (TCSC) algorithm, a time delay algorithm, and a kurtosis algorithm.

17. The system of claim 11, wherein the characteristic is generated based on a binary character sequence constructed by a cosine window function and the vital sign signal.

18. The system of claim 11, wherein the characteristic includes a signal distribution density calculated based on a reconstructed trajectory of the vital sign signal.

19. The system of claim 11, wherein the characteristic includes a kurtosis calculation result.

20. The system of claim 11, the analysing, based on a secondary peak value and a position of the secondary peak before the peak or a secondary peak value and a position of the secondary peak after the peak, a noise detection result of the first manner includes: determining a count of peak(s) in a current window period, a maximum peak and a minimum peak; when the count of peak(s) is greater than or equal to 2, and a difference between the maximum peak and the minimum peak is greater than a predetermined threshold, or the count of peak(s) is less than or equal to 1, determining that the vital sign signal of the current window period contains a noise.
